(19) United States
(12) Patent Application Publication
(10) Pub. No.: US 2022/0225973 A1
(43) Pub. Date: Jul. 21, 2022

(54) FLEXIBLE SURGICAL INSTRUMENTS FOR PERCUTANEOUS AND NEUROSURGICAL APPLICATIONS

(71) Applicant: The Chinese University of Hong Kong, Hong Kong (CN)

(72) Inventors: Shing Shin CHENG, Hong Kong (CN); Kim YAN, Hong Kong (CN); Junyan YAN, Liaocheng (CN)

(21) Appl. No.: 17/151,081

(22) Filed: Jan. 15, 2021

(51) Int. Cl. A61B 17/00; A61B 34/00

(52) U.S. Cl. CPC A61B 17/00; A61B 2034/305; A61B 2017/00867; A61B 34/71

(57) ABSTRACT

The subject invention pertains to percutaneous surgical device with variable stiffness for percutaneous procedures, including but not limited to pericardiocentesis, and flexible neurosurgical devices for procedures, including but not limited to intracerebral hemorrhage evacuation and third ventriculostomy and brain tumor biopsy, and methods of making and using them.

### BACKGROUND OF THE INVENTION

Percutaneous surgical procedures involve the insertion of a surgical instrument via needle-puncture of the skin to access internal tissues, organs, and other biological structures. Pericardiocentesis is an example of percutaneous surgical procedure to drain pericardial effusion surrounding the heart that may cause cardiac tamponade, which is a life-threatening medical condition. The procedure involves the insertion of a needle to the pericardium, which is a fluid-filled sac around the heart under ultrasound guidance. A guidewire is passed through the needle to the pericardium before the needle is removed. Several dilators are inserted and removed over the guidewire to increase the wound size before a drainage catheter is inserted over the guidewire as the drainage channel. The removal of the guidewire with the drainage catheter staying on completes the pericardiocentesis. The procedure carries the risk of serious complications such as cardiac perforation with the needle pointing toward the beating heart during its insertion process, even under ultrasound guidance. Flexible or continuum surgical robots with small diameters have been developed but most of them are laparoscopic surgical instruments. They have not been tested for buckling resistance in percutaneous procedures. Many variable stiffness mechanisms have been adopted to increase the rigidity of continuum surgical robots; however, safe and efficient bendable devices are needed for percutaneous applications.

Intracerebral hemorrhage (ICH) is the deadliest and one of the most common forms of stroke in the world with up to 40% morbidity and mortality rate among all stroke cases. ICH occurs when a blood vessel in the brain ruptures and blood accumulates within the cranial cavity, compressing the neighboring brain region, which in turn is deprived of oxygenated blood supply. While craniotomy is the most widely used technique for surgical evacuation of ICH, large-scale randomized trials have shown only marginal clinical and survival benefits and the high degree of invasiveness potentially offsets its benefits. Minimally invasive techniques using rigid instruments such as a neuroendoscope and stereotactic sheath have been explored as alternatives and have shown to provide promising surgical outcomes but the hematoma evaluation percentage is often limited by the small access channel and the lack of dexterity of the rigid tools. The insertion and manipulation of the straight and rigid neuroendoscope and instruments also potentially cause significant brain manipulation and post-operative trauma. While steerable needles are capable of approaching an ICH through a nonlinear path, their insufficiently dexterous distal motion, small distal curvature, and inability to incorporate ICH evacuation tools have hindered their application in ICH evacuation. Thus, the optimal surgical approach to ICH evacuation remains an open research problem to date. Minimally invasive techniques have shown promise but unsubstantial clinical benefits due to the limited distal dexterity and damage to healthy brain tissues limit their application.

Endoscopic third ventriculostomy (ETV) and tumor biopsy is a minimally invasive brain surgery used to manage hydrocephalus and obtain pineal tumor sample for diagnosis. Long, straight, and semi-rigid instruments are generally inserted through an instrument channel (˜2 mm diameter) of a neuroendoscope to reach structures in the third ventricle floor. They, however, do not allow biopsy of a tumor that is often located at a significant angle to the endoscope insertion direction. Thus, neuroendoscopic instruments, that possess flexible distal segments, can produce distal bending outside the line of sight, and allow the tumor biopsy to be performed without significant manipulation of the neuroendoscope or a second skull incision.

### BRIEF SUMMARY OF THE INVENTION

Provided are flexible surgical devices for percutaneous procedures (e.g. pericardiocentesis), intracerebral hemorrhage evacuation and third ventriculostomy and brain tumor biopsy, and methods of making and using them. The first percutaneous device features a flexible wrist made of a shape memory alloy (SMA) spring which can vary its stiffness through temperature variation. The SMA spring can be heated and thus stiffened to allow needle insertion. At lower temperature, it behaves like a spring backbone that enables large curvature bending, allowing the needle to be pointed away from a healthy tissue and thus minimizing the chance of damage to healthy tissue. Further provided is a heating tube made of nichrome wire and PTFE tube as controlled heat source for the SMA spring. The second neurosurgical device features a straight or pre-curved body and a dexterous independently controlled flexible tip segment. The body can be made of metals, alloys or other super-elastic materials such as Nickel Titanium. The pre-curved body can be translated and rotated, allowing a non-linear insertion path towards the intracerebral target. The flexible tip segment can be made of flexible structures such as a Nickel Titanium tubing with symmetrically or asymmetrically notches. It can be bent using wires/cables in 2 degrees of freedom to allow 3-dimensional workspace coverage. The third neurosurgical device features a straight rigid body and a flexible distal segment that can be bent in a single direction. The distal segment features lateral notches with asymmetric notch patterns and dimensions, allowing bending towards one direction with large bending curvature up to around 150 m⁻¹ and bending angle of around 90° upon cable pulling. The smaller notch height on one side minimizes the mechanical stress during the large bending of the segment and provides a mechanical limit to any undesired bending.

Advantageously, the surgical devices have small enough outer diameter to minimize tissue damage during insertion with a lumen sufficiently large to pass instruments through. Further, the bendable distal tips of the devices facilitate the distal dexterity of the devices and enable wristed motion for safe and effective use of the devices of the invention for pericardiocentesis, intracerebral hemorrhage evacuation, and third ventriculostomy and brain tumor biopsy. The devices can be used with suitable imaging modalities, including ultrasound, computed tomography, endoscopy, and magnetic resonance imaging, to facilitate image-guided surgical procedures.

### BRIEF DESCRIPTION OF THE DRAWINGS

FIG. 1A shows a magnified view of the distal fixture of the structure of the wristed percutaneous device comprising a needle, a flexible wrist, and a rigid arm. FIG. 1B shows a magnified view of the proximal fixture of the wristed percutaneous device. FIG. 1C shows a magnified view the cross-section of the rigid arm of the wristed percutaneous device. FIG. 1D shows a magnified view of the silicone sheath that fits over the flexible wrist. FIG. 1E shows the structure of the wristed percutaneous device comprising a needle, a flexible wrist, and a rigid arm.

FIG. 2 shows the flexible wrist of the percutaneous robot consisting of a SMA spring, distal fixture (DF), and proximal fixture (PF).

FIG. 3 shows a heating tube made of a PTFE tube wound with Nickel Chromium wire on its external surface.

FIG. 4 shows a robotic platform used for experiments comprising a linear actuator, robot, and rigid base.

FIG. 5A shows the theoretical and experimental changes in bending angle in response to the cable displacement. FIG. 5B shows the theoretical and experimental position changes in response to the cable displacement.

FIG. 6 shows the temperature changes of an SMA spring and a wrist surface when the SMA was heated to 48° C. before cooling down to 37.5° C. inside a water bath where line (a) indicates the time when the robot insertion into the water bath began and line (b) indicates the end of the device insertion process.

FIG. 7A shows the device insertion and bending to the left and right in 15% gelatin. FIG. 7B shows the device insertion and bending to the left and right in 20% gelatin.

FIG. 8A shows ultrasound image during the device insertion process with the bright white line indicating the border between two phantom layers of different stiffnesses. FIG. 8B shows ultrasound image of the device after insertion and passing of the two phantom layers in a straight configuration. FIG. 8C shows ultrasound image of the device when it was stopped after insertion. FIG. 8D shows ultrasound image of the device when its distal tip was bent upward.

FIG. 9 shows an illustration of the proposed neurosurgical device with a pre-curved body and flexible bendable tip during ICH evacuation (left) and a prototype of the proposed device consisting of an outer tube, a pre-curved inner tube and a flexible tip (right).

FIG. 10 shows a graphical comparison of the surgical approach using a straight trajectory (top) and the proposed non-linear or curved trajectory (bottom) where the curved insertion trajectory offers more skull incision options.

FIG. 11A shows a CAD drawing of a flexible tip of the proposed device. FIG. 11B shows an A-A cross section for demonstrating the cable routing method. FIG. 11C shows a 2-DoF symmetric rectangular notch pattern.

FIG. 12 shows an actuation unit of the robotic version of the proposed neurosurgical device.

FIG. 13A shows a laser micromachining of the flexible tip. FIG. 13B shows a stereoscopic photo of the flexible tip.

FIG. 14 shows an experimental set-up for kinematics validation and control framework evaluation of the proposed neurosurgical device.

FIG. 15A shows a plot of the bending angle against the cable displacement. FIG. 15B shows the changes in tip position in response to cable displacement.

FIG. 16A shows X, Y, and Z coordinates of a distal end of the cannula body and the plotted against time. FIG. 16B shows the bending angle of the cannula tip plotted against time.

FIG. 17 shows the insertion of the proposed neurosurgical device into the gelatin brain phantom and the subsequent flexible tip bending in the 2 degrees of freedom of pitch and yaw.

FIG. 18 shows a CAD illustration of the proposed endoscopic neurosurgical device with a straight body and a flexible bendable tip with unidirectional asymmetric notches capable of single direction bending at large bending curvature.

FIG. 19A shows a CAD illustration of the flexible tip of the proposed endoscopic neurosurgical device with lateral notches of asymmetric patterns and dimensions. FIG. 19B shows a CAD illustration of the flexible tip of the proposed neurosurgical device with a surgical forceps as its instrument.

FIG. 20A shows an endoscopic neurosurgical device with flexible distal segment. FIG. 20B shows a magnified view of the flexible distal segment with a series of lateral notches with asymmetric patterns and dimensions.

FIG. 21 shows a robotic control interface for the endoscopic neurosurgical instrument.

FIG. 22 shows an experimental setup to evaluate the mechanical properties of the endoscopic neurosurgical instrument.

FIG. 23 shows the changes in bending angle in response to the changes in cable tension during both loading and unloading stages.

FIG. 24 shows the changes in bending angle in response to the changes in cable displacement during both loading and unloading stages.

### DETAILED DISCLOSURE OF THE INVENTION

Provided are a percutaneous device with variable stiffness for percutaneous procedures, including pericardiocentesis, and flexible neurosurgical devices appropriate for various neurosurgical procedures including intracerebral hemorrhage evacuation and third ventriculostomy and brain tumor biopsy, and methods of making and using them. The first device features a flexible wrist made of a shape memory alloy (SMA) spring which can vary its stiffness through temperature variation. The SMA spring can be heated and thus stiffened to allow needle insertion. At lower temperature, it behaves like a spring backbone that enables large curvature bending, allowing the needle to be pointed away from a healthy tissue and thus minimizing the chance of damage to healthy tissue. Further provided is a heating tube made of nichrome wire and PTFE tube as controlled heat source for the SMA spring. The second device features a straight or pre-curved body and a dexterous independently controlled flexible tip segment. The body can be made of metals, alloys or other super-elastic materials such as Nickel Titanium. The pre-curved body can be translated and rotated, allowing a non-linear insertion path towards the intracerebral target. The flexible tip segment can be made of flexible structures such as a Nickel Titanium tubing with symmetrically or asymmetrically notches. It can be bent using wires/cables in 2 degrees of freedom to allow 3-dimensional workspace coverage. The third device features a straight rigid body and a flexible distal segment that can be bent in a single direction. The distal segment features lateral notches with asymmetric notch patterns and dimensions, allowing bending towards one direction with large bending curvature up to around 150 m⁻¹ and bending angle of around 90° upon cable pulling. The smaller notch height on one side minimizes the mechanical stress during the large bending of the segment and provides a mechanical limit to any undesired bending.

Advantageously, the surgical devices enable distal dexterity of their tip for precise targeting and dexterous manipulation of a structure and/or cavity inside a body of a subject. The devices have small enough outer diameter to minimize tissue damage during insertion while a lumen sufficiently large to pass instruments through. Further, the bendable distal tips of the devices facilitate the distal dexterity of the devices and enable wristed motion for safe and effective use of the devices of the invention for percutaneous procedures, including but not limited to pericardiocentesis, and neurosurgical procedures, including but not limited to intracerebral hemorrhage evacuation and third ventriculostomy and brain tumor biopsy. The devices can be used with suitable imaging modalities, including ultrasound, computed tomography, endoscopy, and magnetic resonance imaging, to facilitate image-guided surgical procedures.

In some embodiments, the devices of the invention comprise a percutaneous device comprising a needle, a flexible wrist with variable stiffness, and a rigid arm. Advantageously, the percutaneous device of the invention are capable of needle intervention without buckling and due to the bendable wrist can divert the needle from structures that are not to be damaged during device operation inside a body of a subject. Therefore, the device of the invention not only avoids the risk of damaging a structure close to the operation area of the device but also enables the passage of a guidewire into a space or cavity inside a subject for surgical treatment of a condition in the subject.

In some embodiments, the device of the invention is a neurosurgical device for removal of intracerebral lesions, including intracerebral hemorrhage.

In some embodiments, the device of the invention is a flexible neuroendoscopic instrument for endoscopic ventriculostomy and/or tumor biopsy.

In preferred embodiments, the percutaneous device for pericardiocentesis comprises a needle, a flexible wrist, and a rigid arm and the bendable wrist diverts the needle from pointing at a heart as it approaches a pericardial space. The device, thus, not only avoids the risk of cardiac perforation but also facilitates the passage of a guidewire into the pericardial space.

In some embodiments, the wristed percutaneous pericardiocentesis device of the invention allows large curvature distal bending that minimizes the risk of cardiac perforation and simultaneously facilitates the passage of guidewire into the pericardial cavity.

In some embodiments, the device of the invention comprises a shape memory alloy (SMA) spring-based wrist that can be stiffened to facilitate the robot insertion into the biological tissues and can be used like a flexible joint that allows large curvature distal bending.

In preferred embodiments, the device of the invention comprises a miniature SMA spring that is robustly integrated with the other parts of the robot.

In some embodiments, the device comprises a novel heating tube with a small diameter of about 1 mm wrapped with NiCr wires to enable a controlled heating of the SMA spring.

Further provided are a stiffness model and a kinematics model to experimentally validate the performance of the devices of the invention. Advantageously, using these models it was demonstrated that the robot of the invention was sufficiently stiff to be inserted into gelatin phantoms simulating the lipid and muscle layers. Further, the models were used to determine a threshold transformation temperature of the SMA spring that eliminates any safety concern for use in a subject. The surface temperature of the robot wrist was evaluated to ensure that the SMA heating mechanism did not induce thermal damage to tissues.

In some embodiments, the device of the invention comprises a closed-loop controller for the percutaneous robotic device by integrating ultrasound image feedback.

In some embodiments, an SMA spring is used as both the stiffening material and the flexible wrist structure.

Advantageously, the wristed surgical device of the invention is capable of both percutaneous insertion and large curvature distal bending for a safer pericardiocentesis.

In some embodiments, novel fabrication methods are provided that ensure highly reliable connection between SMA spring and other metallic structures in a dynamic condition.

Further provided are kinematics and stiffness models and methods for verification of the performance of the robots.

In some embodiments, the device of the invention is capable of performing percutaneous insertion towards the heart and produces a distal bending in the pericardial space. In specific embodiments, the robot of the invention has a maximum outer diameter of about 4 mm. In further specific embodiments, the inner diameter of the robot of the invention is large enough to accommodate a tubing of about 0.5 mm diameter and a guidewire of around 200 mm. In further embodiments, the tubing of the device of the invention allows drainage of fluids through suctioning once the robot reaches the pericardial cavity and the guidewire is passed through the robot to facilitate the insertion of a cannula during a procedure.

In preferred embodiments, the robot is able to bend at its distal tip to steer the tip away from the beating heart and facilitate the guidewire to stay inside the pericardial space after passing through the robot lumen. In further preferred embodiments, the flexible tip of the robot of the invention has a large bending curvature (>150 m⁻¹) to allow 90° bending with less than 10 mm bending length to accommodate to a pericardial effusion depth between about 10 mm to about 20 mm.

In some embodiments, the device of the invention comprises a distal fixture comprising a needle hub on one end and four prongs on the other end to interface with the needle and the SMA spring, respectively. In some embodiments, the device comprises a proximal fixture comprising four prongs on one end and four straight legs on the other end to interface with the SMA spring and the rigid arm, respectively. In further embodiments, the device comprises an elastic sheath made of silicone wrapped around the SMA spring as a protective cover that separates the cables and SMA spring from the surrounding biological tissues.

In some embodiments, the rigid arm of the robot comprises a hollow tube with a length of about 80 mm and a lumen of about 1.4 mm diameter and accommodates a heat shrink tube and a Polytetrafluoroethylene (PTFE) tube. The PTFE tube allows drainage of the pericardial fluid and passage of a guidewire into the pericardial space. In some embodiments, there are four equidistantly-spaced rings of about 1 mm diameter at two ends of the rigid arm to hold four long rigid stainless steel tubes of about 0.9 mm outer diameter along the periphery of the lumen of the rigid arm. In some embodiments, the long tubes act as channels for the passage of actuation cables. In some embodiments, the actuation cables can have a diameter of around 0.21 mm diameter. Advantageously, the channels serve as dedicated pathways for the cables, inhibiting any issue of cable tangling and more importantly, keeping the center of the lumen clear.

In some embodiments, the neurosurgical instrument of the invention comprises a flexible sheath made from metal or polymer, wherein the sheath is integrated with the instrument to serve as actuation tendon or rod routing channel and holds sensors, including but not limited to Fiber Bragg Grating (FBG).

In some embodiments, the SMA spring in the flexible wrist of the device of invention has a length of around 8 mm, an outer diameter of 2.5 mm, spring wire diameter of 0.75 mm, and a lumen of 1 mm. In further embodiments, the SMA spring can have a length from about 20 mm to about 2 mm, about 19 mm to about 3 mm, about 18 mm to about 4 mm, about 17 mm to about 5 mm, about 16 mm to about 6 mm, about 15 mm to about 7 mm, about 14 mm to about 8 mm, about 13 mm to about 9 mm, about 12 mm to about 10 mm.

In some embodiments, the SMA spring can have an out diameter of about 4 mm to about 1 mm, about 3.5 mm to about 1.2 mm, about 3 mm to about 1.5 mm, about 2.8 mm to about 1.8 mm, about 2.6 mm to about 2 mm, or about 2.8 mm to about 2.2 mm.

In some embodiments, the spring wire can have a diameter of 1 mm to about 0.1 mm, about 0.9 mm to about 0.2 mm, about 0.8 mm to about 0.4 mm, about 0.75 mm to about 0.5 mm, about 0.7 mm to about 0.6 mm.

In some embodiments, the SMA spring can have a lumen of about 1.5 mm to about 0.1 mm, about 1.2 mm to about 0.2 mm, about 1 mm to about 0.4 mm, about 0.9 mm to about 0.5 mm, or about 0.8 mm to about 0.6 mm.

In preferred embodiments, the spring comprises a material that converts its material phase between the low stiffness martensite and the high stiffness austenite through temperature variation. For example, heating the material of the spring beyond the austenite finish temperature of around 45° C. raises its stiffness to facilitate the percutaneous needle insertion process while cooling the spring material down below the martensite finish temperature allows it to have reduced stiffness and behave like a flexible spring.

In some embodiments, the wrist of the device of the invention is capable of a 2-degree of freedom (DoF) bending using a cable-driven mechanism with one antagonistic cable pair responsible for each of the pitch and yaw DoFs.

In some embodiments, the four stainless steel actuation cables terminate at the distal fixture (DF) and are equiradially and equidistantly spaced around the SMA spring segment. In further embodiments, the actuation cables are passed through the peripheral channels in the proximal fixture (PF) and the rigid tube to be connected to the actuation unit or control interface.

In some embodiments, the wrist of the device of the invention can be bent to 90°, allowing the robot tip to reach the desired workspace in the pericardial cavity.

In some embodiments, the device of the invention comprises a heating element in the form of a compact tube that is inserted into the lumen of the SMA spring and acts as a controlled heat source. In some embodiments, the heating tube is made of a PTFE tube wound with NiCr wire on its external surface. In some embodiments, the heating tube consists of two parts, namely the high resistance NiCr 2080 enameled coil of about 30 mm diameter and 80 mm length, and a PTFE tube of 1 mm diameter. In further embodiments, a small portion of the enamel is removed from the two ends of the NiCr wire which are then connected to two copper electric wires of 48 AWG by soldering. In further embodiments, a thin layer of insulating paint ion clothing is painted on the soldering joints to avoid the risk of short circuit.

In some embodiments, the high resistance NiCr 2080 enameled coil has a diameter of about 40 μm to about 5 μm, about 38 μm to about 7 μm, about 36 μm to about 10 μm, about 34 μm to about 12 μm, about 32 μm to about 14 μm, about 30 μm to about 16 μm, about 28 μm to about 18 μm, about 26 μm to about 20 μm, or about 24 μm to about 22 μm.

In some embodiments, the high resistance NiCr 2080 enameled coil has a length of about 100 mm to about 20 mm, about 95 mm to about 25 mm, about 90 mm to about 30 mm, about 85 mm to about 35 mm, about 80 mm to about 30 mm, about 78 mm to about 35 mm, about 75 mm to about 40 mm, about 70 mm to about 45 mm, or about 68 mm to about 50 mm.

In some embodiments, the distal section of the external surface of the PTFE tube, which was also used as the fluid and guidewire channel, is sanded with a sandpaper to increase its surface roughness. A high temperature medical super glue is applied to glue the NiCr wire on the tube. In preferred embodiments, the NiCr wire coil is wound with a relatively constant pitch for 8 mm distance along the tube, ensuring that the SMA spring is heated uniformly along its entire segment. Further, a heat shrink tube is slid over the PTFE tube and the electrical wires and acts as a protective layer for the wires. In some embodiments, the NiCr wire is folded in the middle in half before being wound around the sanded part of the PTFE tube to allow both the positive and negative terminals of the electric wires to exit from the proximal part of the PTFE tube.

In preferred embodiments, the SMA spring is strongly and rigidly connected to both the rigid arm and the needle. Therefore, the distal fixture (DF) and proximal fixture (PF) holding the SMA springs are designed to have multiple prongs and legged features to increase the surface area of connection. The DF and PF also have small channels for the termination and passage of the actuation cables, and a central lumen for the passage of the heating element.

Also provided is a robotic platform comprising the percutaneous robotic device, a rigid base, and up to four linear actuators. In some embodiments, the robot is attached to a rigid base that contains a few pulleys for cable routing. In some embodiments, four cables are connected to the two or four linear actuators to pretension the robot wrist and to actuate the robot in one degree of freedom (DoF).

In some embodiments, phantom models are provided to test the ability of the device of the invention to perform percutaneous procedures and distal bending at its wrist. The models comprise different gelatin concentrations, including but not limited to phantoms of 15% and 20% by weight gelatins to simulate biological tissues with different stiffness.

In some embodiments, the methods of the invention comprise heating an SMA spring of the invention to and maintain at around 48° C. while the actuation cables are pretensioned and maintained at around 1N.

In further embodiments, the methods comprise insertion of the device into the phantom by a linear actuator at a speed of 0.125 mm/s with, e.g, insertion of about 30 mm of the device into the phantoms. Advantageously, the device of the invention is capable of being inserted into the gelatin phantoms of different stiffness along a straight path without any noticeable sign of buckling. In a further embodiment, the methods comprise stopping the heating after the insertion process such that the SMA spring cools down and the device tip is bent by 90° for a few cycles by actuating the cables connected to linear actuators on the proximal end. Advantageously, the actuators are powerful enough to bend the wrist by the required bending angle even when the wrist is still in the phantom models. Thus, the device of the invention is capable of producing distal bending even when a cavity, such as, e.g., a pericardial cavity is only large enough to accommodate the needle but not the entire device wrist, where the wrist can remain in a stiffer biological tissue.

Further provided are devices for neurosurgical procedures, including but not limited to, intracerebral hemorrhage (ICH) evacuation.

In specific embodiments, the device comprises of a straight tube and a flexible tip, allowing a straight trajectory towards the neurosurgical lesion (e.g. ICH) and dexterous distal bending for enhanced peripheral lesion (e.g. ICH) evacuation.

In specific embodiments, the device comprises of a straight outer tube, a pre-curved inner tube and a flexible tip, allowing a curved or non-linear trajectory towards the neurosurgical lesion (e.g. ICH) and dexterous distal bending for enhanced peripheral lesion (e.g. ICH) evacuation.

In some embodiments, the device of the invention is created by cutting lateral notches of various shapes on a super-elastic Nitinol tube, which becomes sufficiently flexible to be bent by cables. Advantageously, the device of the invention combines a pre-curved tube and a flexible tip to achieve a curved entry trajectory and distal dexterity for neurosurgical lesion aspiration or removal. This allows avoidance of critical structures along the normally straight trajectory between the skull incision and the neurosurgical lesion, and satisfies the dexterous distal motion with enhanced workspace. This also provides more incision options and avoids incision close to facial features.

In preferred embodiments the device of the invention is an integrated design of a pre-curved tube and a flexible tip that are monolithically fabricated in a single tube. Advantageously, this combination enables motion decoupling between the body and its tip and provides superior distal dexterity. Further, the device of the invention is actuated at its distal end with around 90° bending capability in any direction providing superior reach and enabling clinically meaningful neurosurgical lesion decompression while minimizing the disruption to healthy brain tissue during the lesion aspiration/removal process.

In preferred embodiments, the dimensions of the device are large enough to house a suction tube or lesion removal instrument of sufficiently large diameter that facilitates lesion aspiration or removal while remaining small enough to reduce trauma to healthy brain tissue.

In preferred embodiments, the device of the invention has a total of at least five active degrees of freedom (DoF) including, a translation DoF of the outer tube, a rotation DoF of the inner cannula, a translation and a rotation of the device body and a 2-DoF bending of the device tip. In some embodiments, the outer tube can be a multi-tube concentric tube pair.

In some embodiments, the device of the invention has a total of 5 active degrees of freedom (DoF) including a translation of the outer straight tube, translation and rotation of the device body and a 2-DoF bending of the device tip.

In some embodiments, the cable-driven device tip can be independently actuated, thus minimizing the lateral motion of the device body and disruption to the healthy brain tissues during its motion.

In some embodiments, the device body comprises a straight outer tube with an outer diameter D=3.0 mm and an inner diameter d=2.6 mm, and a pre-curved inner tube of an outer diameter D=2.2 mm and an inner diameter d=2.0 mm, both of which are made of the biocompatible super-elastic Nitinol.

In some embodiments, the length of the outer tube is 100 mm while that of the inner tube is s220 mm and the outer tube provides a short straight channel extending from the base platform attached to the skull, through the skull incision, to the brain surface, while the inner tube, with a pre-curved shape, covers a curved region based on its pre-curved shape once it is extended out of the constraining outer tube. In some embodiments, the radius of curvature of the pre-curved inner tube is 55 mm with a 45° bending angle. Advantageously, the curvature parameters can be customized depending on the individual neurosurgical lesion case and are only limited by the approximately 8% sustainable recoverable strain of the Nitinol tube.

In some embodiments, the flexible tip of the device is generated by creating notches and therefore introducing flexibility in an originally rigid straight super-elastic Nitinol tube.

In preferred embodiments, a symmetric rectangular notch pattern is used due to the large curvature required in the design with a 90° bending angle with only approximately 22 mm bending length. In some embodiments, a total of N=10 pairs of notches are equally and alternately distributed in two orthogonal planes on the Nitinol tube.

In some embodiments, the device of the invention utilizes cable-driven mechanism to actuate the flexible tip with two pairs of Stainless steel wire ropes responsible for pitch and yaw bending, respectively, that are routed along the periphery of the tube inside its lumen and terminated at the distal end of the tube.

In preferred embodiments, each actuation cable is constrained within the tiny space formed between a thin super-elastic Nitinol wire crossing and the tube inner wall, where each wire of about 0.15 mm diameter is inserted between a pair of opposite holes on the tube wall near the routing path of the cable. For each actuation cable, a pair of these holes is generated every two notches along the flexible tip to ensure the cable is robustly routed on the periphery of the tube throughout the bending process.

In some embodiments, an actuation unit is comprised of a cable control module and a device translation module with the inner tube of the device directly connected to the cable control module, which is responsible for the 2-DoF bending motion of the flexible tip. The device translation unit comprising a linear module and a DC motor translates the cable control module and, thus, inserts and retracts the inner tube. Once the inner tube reaches the targeted point in the neurosurgical lesion, the cable control module is activated to sweep around the lesion while performing hematoma aspiration.

In some embodiments, every component of the device, including but not limited to, the straight outer tube, the pre-curved tube, the flexible tip, the actuation unit, the cable control module, the device translation module, actuators, sensors, and the skull attachment unit, is made of magnetic resonance imaging (MRI)-compatible (MRI-conditional or MRI-safe) materials. This allows the device to be implemented in the MM scanner for manual or robotic surgical procedures.

Further provided is a kinematic model that maps from the actuator space of the tube translation and cable displacement to the task space of the device tip position to validate device performance. Also provided is a cable elongation model. Advantageously, the device of the invention is able to track discrete positions in its workspace under an optimization-based controller utilizing the robot Jacobian, with a root mean square error of 1.5 mm.

Further provided is a flexible neuroendoscopic device for third ventriculotomy and tumor biopsy.

In some embodiments, the endoscopic device features asymmetric notch patterns and dimensions in its distal segment where the asymmetric notch patterns allow distal bending in a single plane while the asymmetric notch dimensions allow large angle bending in one direction with minimal structural stress.

In some embodiments, the device comprises a straight rigid body and a flexible distal segment. In preferred embodiments, the flexible distal segment of the device features around 20 asymmetric lateral notches with the left notches having larger notch height than the right notches, allowing bending towards one (left) direction with large bending curvature up to around 150 m⁻¹ and a bending angle of around 90° upon cable pulling. The small notch height on the right side minimizes the structural stress during the large bending of the segment towards the left side and provides a mechanical limit to inhibit large bending angle towards the right side.

In some embodiments, the flexible distal segment of the device is manufactured suing a femtosecond laser micromachining system to remove rectangular lateral notches with asymmetric patterns and dimensions at the distal section of a Nickel Titanium (Nitinol) tubing to introduce the distal flexibility to an originally rigid tube.

In specific embodiments, the geometrical parameters of each notch, including the notch shape, lateral cutting depth, distance between consecutive notches, notch height, are carefully selected to achieve the desired bending stiffness and bending curvature of the instrument.

In some embodiments, a stainless steel actuation cable with a diameter of 0.15 mm is terminated at the distal end of the flexible segment and routed along the lumen of the tubing before being connected to a control interface at the proximal end.

In some embodiments, surgical instrument, including but not limited to biopsy forceps, that is either actively or passively actuated is integrated at the distal end of the instrument to perform tumor biopsy at locations outside the line of sight due to the capability of the instrument to bend its distal segment.

In some embodiments, an actuation cable is terminated at a distal end of the flexible instrument, routed along the lumen of the instrument, and connected to a motorized actuation setup comprising a motor, encoder, and a cable tension sensor.

In some embodiments, the actuation setup is replaced by a custom-designed user control interface for manual operation.

Advantageously, when the device is bent to about 70°, the cable tension required is high proving a sufficiently high stiffness of the device tip for motion in the brain and tissue manipulation. Further, when the device is bent to about 70° the cable is displaced for less than 2 mm demonstrating the use of a small amount of cable displacement of the device to satisfy the required bending angle.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Materials and Methods

### Example 1—Design and Fabrication

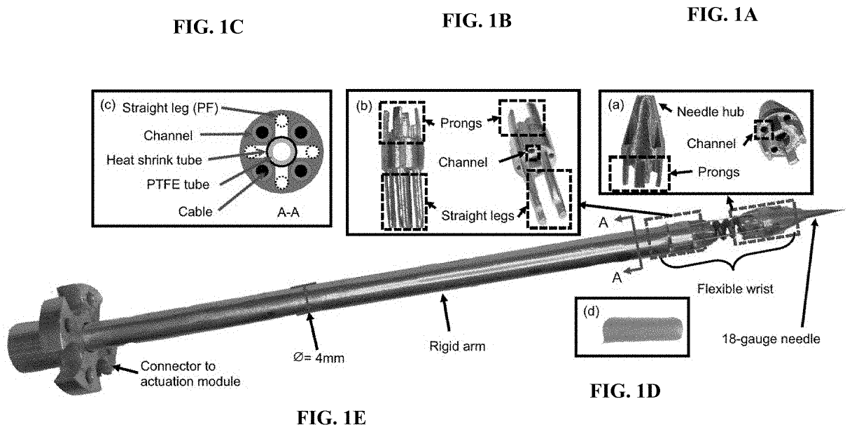

A steerable percutaneous device was developed as a surgical robot to perform robotic percutaneous insertion towards the heart and produce distal bending in the pericardial space. The following design requirements were met by the percutaneous robot generated: 1) The robot had a maximum outer diameter of 4 mm, matching the diameter of the dilator that is used in existing procedures after the initial needle puncturing. 2) The inner diameter of the robot was large enough to accommodate a tubing of 0.5 mm diameter and a guidewire of around 0.2 mm. The tubing allowed drainage of fluids through suctioning once the robot reached the pericardiac cavity while the guidewire was passed through the robot to facilitate the insertion of a cannula in the final step of the procedure. 3) The robot was able to bend at its distal tip to steer the tip away from the beating heart and facilitate the guidewire to stay inside the pericardial space after passing through the robot lumen. The flexible tip of the robot had a large bending curvature (>150 m⁻¹) to allow 90° bending with less than 10 mm bending length to cater to pericardial effusion depth between 10 mm-20 mm. The percutaneous robot can be considered as a combination of a 12-Fr dilator and a 18-gauge needle, with the largest outer diameter being 4 mm. As seen in FIG. 1E the robot consisted of three major parts, namely a 18-gauge bevel tip stainless steel needle, a flexible wrist, and a rigid arm. The wrist was composed of an SMA spring and two fixtures on two ends of the spring, namely the distal fixture (DF) and proximal fixture (PF). The DF, as shown in two views in FIG. 1A, featured a needle hub on one end and four prongs on the other end to interface with the needle and the SMA spring, respectively. The PF, shown in two views in FIG. 1B, featured four prongs on one end and four straight legs on the other end to interface with the SMA spring and the rigid arm, respectively. An elastic sheath made of silicone was wrapped around the SMA spring as a protective cover that separated the cables and SMA spring from the surrounding biological tissues (FIG. 1D).

The rigid arm of the robot was a hollow tube with a length of 80 mm and a 1.4 mm diameter lumen that was sufficiently large to accommodate a heat shrink tube and a Polytetrafluoroethylene (PTFE) tube, as shown in FIG. 1D. The PTFE tube allowed drainage of the pericardial fluid and passage of guidewire into the pericardial space. It also functioned as a heating element (see EXAMPLE 2). There were four equidistantly-spaced 1 mm diameter rings at two ends of the rigid arm to hold four long rigid stainless steel tubes of 0.9 mm outer diameter along the periphery of the lumen of the rigid arm. The long tubes acted as channels (refer to FIG. 1C) for the passage of actuation cables with 0.21 mm diameter. This constituted a low-cost yet effective solution to compensate for the inability of commonly accessible manufacturing techniques to produce sub-millimeter diameter channels with large aspect ratio (>100). These channels served as dedicated pathways for the cables, inhibiting any issue of cable tangling and more importantly, keeping the center of the lumen clear.

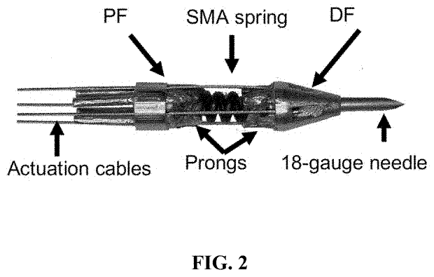

As shown in FIG. 2, the SMA spring in the flexible wrist had a length of around 8 mm, an outer diameter of 2.5 mm, spring wire diameter of 0.75 mm, and a lumen of 1 mm. It could convert its material phase between the low stiffness martensite and the high stiffness austenite through temperature variation. Heating it beyond the austenite finish temperature of around 45° C. raised its stiffness to facilitate the percutaneous needle insertion process while cooling it down below the martensite finish temperature allowed it to have reduced stiffness and behave like a flexible spring. The wrist was capable of a 2-degree of freedom (DoF) bending using a cable-driven mechanism with one antagonistic cable pair responsible for each of the pitch and yaw DoFs. The four stainless steel actuation cables, terminated at the DF, were equiradially and equidistantly spaced around the SMA spring segment. They were passed through the peripheral channels in the PF and the rigid tube to be connected to the actuation unit. The wrist could be bent to 90°, allowing the robot tip to reach the desired workspace in the pericardial cavity.

### Example 2—Heating Tube

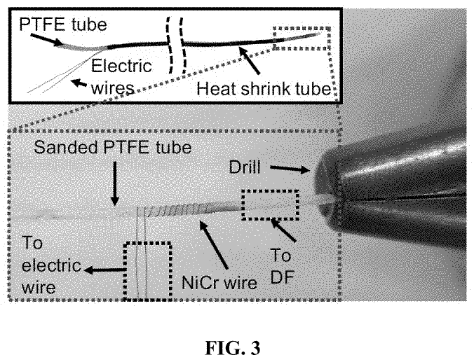

Methods that could heat and stiffen the SMA spring were limited due to the small dimension of the robot. Because a soldering joint between a Nitinol and a copper electrical wire was relatively weak; it was highly challenging to create a soldering spot joint that did not increase the overall diameter of the spring, and routing a NiCr wire along the SMA wire turns of such a miniature SMA spring uniformly could lead to non-uniform distribution of the NiCr wires and therefore non-uniform heating along the SMA spring. Thus, a different design was generated. A heating element in the form of a compact tube that could be inserted into the lumen of the SMA spring and act as a controlled heat source was generated. The heating tube made of a PTFE tube wound with NiCr wire on its external surface is shown in FIG. 3.

As shown in FIG. 3, the heating tube consisted of two parts, namely the high resistance NiCr 2080 enameled coil of 30 mm diameter and 80 mm length, and a PTFE tube of 1 mm diameter. A small portion of the enamel was removed from two ends of the NiCr wire, which were then connected to two copper electric wires of 48 AWG by soldering. A thin layer of insulating paint ion clothing was painted on the soldering joints to avoid the risk of short circuit.

The distal section of the external surface of the PTFE tube, which was also used as the fluid and guidewire channel, was sanded with a sandpaper (1500 Grit) to increase its surface roughness, allowing high temperature medical super glue (435, LOCTITE®, Germany) to bind on more easily in the next step. The PTFE tube was held by a drill that assisted the winding process. The NiCr wire was folded in the middle in half before being wound around the sanded part of the PTFE tube. This allowed both the positive and negative terminals of the electric wires to exit from the proximal part of the PTFE tube, as shown in the inset of FIG. 3. The glue was applied carefully to ensure the NiCr wire always stayed on the tube. The NiCr wire coil was wound with a relatively constant pitch for 8 mm distance along the tube, ensuring that the SMA spring was heated uniformly along its entire segment. A heat shrink tube was slid over the PTFE tube and the electrical wires and acted as a protective layer for the wires, as shown in FIG. 3.

### Example 3—Fabrication of the Robot

The fabrication of the robot, which consisted of many miniature components, was a highly complex process. This was mainly due to the difficulty in fusing the SMA spring material, Nickel Titanium (Nitinol), with other metals and in structurally connecting a spring with other cylindrical structures. These challenges were compounded by the need to be structurally robust during both robot insertion into biological tissues and active bending. Besides, the need to heat the miniature SMA spring using hardware that did not further increase the diameter of the robot also complicated the overall design. Consequently, a few fabrication and assembly approaches that ensured the structural robustness of the robot were employed.

Because the SMA spring was the most critical component in the design since it allowed smooth percutaneous insertion in its stiff state and omnidirectional bending in its flexible state, it was critical that the SMA spring was strongly and rigidly connected to both the rigid arm and the needle. Therefore, the distal fixture (DF) and proximal fixture (PF) holding the SMA springs were designed to have multiple prongs and legged features to increase the surface area of connection. The DF and PF also had tiny channels for the termination and passage of the actuation cables, and a central lumen for the passage of the heating element. The DF and PF were fabricated using a combination of wax 3D printing and lost wax casting that supported feature resolution of down to 25 mm. The SMA spring was held between the two fixtures by slightly bending the four prongs of each fixture towards the spring. While the SMA was secured in its position with the bending prongs, this connection was not strong enough to sustain repeated bending cycles. To improve the structural robustness of the flexible wrist, a three-step approach was adopted. First, tiny copper strips were used to fill the relatively visible gaps between the SMA and the prongs. Second, the copper strips, the SMA and the fixture were soldered together using a solder paste (KZ-1513, Kellyshun®), leading to the formation of small solder granules that filled up the gaps. Third, a thin layer of watery glue was applied to fill any remaining gaps, thus acting as an additional support and smoothing the joint surface. The rigid arm was also manufactured using lost wax casting, which allowed the creation of a central lumen of around 100 aspect ratio. Each of the four long tubes was inserted between the rings in the arm lumen and fixed in its position using glue. The elastic sheath was made using dip molding with silicone of 50 shore A hardness.

The assembly of the robot started by inserting the stainless steel channels into the rigid arm through the rings. After the connection of DF, PF and SMA spring together, the PF straight length was inserted into the rigid arm. The heating tube was passed through the center lumen of the rigid arm, DF and SMA spring which was then connected to the PF using glue. The actuation cables were passed through the channels and terminated and DF. The silicone sheath was stretched and slid onto the wrist before the needle was secured on the needle hub of the DF using glue. Vision markers were placed on the DF and PF of the robot during the stiffness and kinematics experiments.

### Example 4—In Vitro Performance Test of the Robot

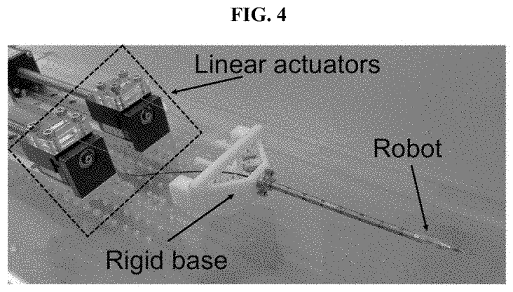

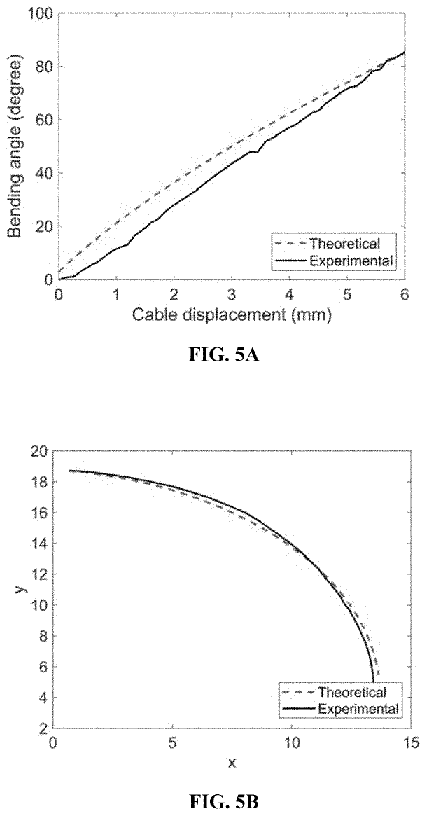

Using gelatin phantoms, the kinematics of the robotic platform were validated, the surface temperature evaluated, and the percutaneous procedure demonstrated. The robotic platform shown in FIG. 4 consisted of the robot, a rigid base, and two linear actuators. The robot was attached to a rigid base that contained a few pulleys for cable routing. In the experiments, only two cables, connected to the two linear actuators were used to pretension the robot wrist and to actuate the robot in one degree of freedom (DoF). The theoretical and experimental changes in bending angle and the theoretical and experimental position changes in response to the cable displacement are shown in FIG. 5A and FIG. 5B.

### Example 5—Verification of a Kinematics Model

In the kinematics verification experiments, the vision tracker was used to track continuously the positions of the markers that were placed on the DF and PF of the robot. One cable was pulled by the linear actuator by 5 mm while the antagonistic cable was released by the same displacement. The bending angle of the robot was calculated from the relative positions of the two markers on the robot. As shown in FIG. 5A, the experimental results for the bending angle matched well with the theoretical data. The largest error of 11° occurred when the cable displacement was small, likely due to the existence of cable slack. When comparing the theoretical and experimental tip positions in FIG. 5B, the mean errors in X and Y positions were 0.4118 mm and 0.1786 mm, respectively, showing the accuracy of the kinematics model within the motion range of the robot.

### Example 6—Flexible Wrist Surface Temperature Evaluation

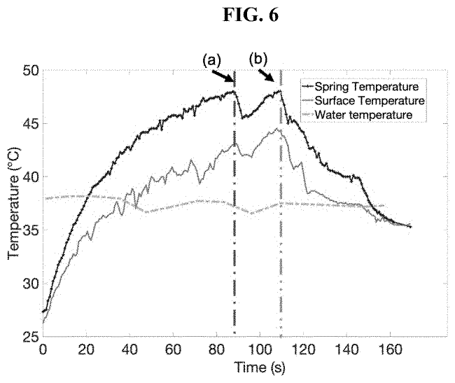

Since the robot was designed to be inserted into the human body, it was critical that the temperature was maintained at a safe level. An experiment was performed to measure the temperatures of both the SMA spring and the surface temperature of the robot wrist when the robot was inserted into a water bath maintained at human body temperature. Temperature sensors (Thermistor NTC 10k Bead, TE Connectivity, Switzerland) were attached to the SMA spring and the external surface of the silicone sheath to record their temperatures throughout the experiment. During the experiment, the SMA spring was heated to 48° C., which is above the austenite finish temperature of 45° C., and inserted into the water bath at a slow speed. A temperature controller was used to ensure the SMA spring would not be heated beyond 48° C. After around 25 s, the heating was stopped, indicating the end of the percutaneous insertion process. The SMA spring was allowed to cool down while the robot was still in the water bath. As shown in FIG. 6, the surface temperature of the robot wrist was always around 5° less than the SMA spring temperature and reached 43° C. at one point. While this was similar to the temperature threshold for thermal damage to normal tissues, a few seconds of contact between the tissue and the robot at 43° C. did not cause any tissue damage. Based on FIG. 6, it took around 50 s for the SMA spring to drop to the equilibrium temperature of 37.5° C. The cooling time was acceptable, considering that the robot did not have any active cooling mechanism. Cooling mechanisms are added in further robotics platforms.

### Example 7—Evaluation of Robot Insertion and Distal Bending Capability

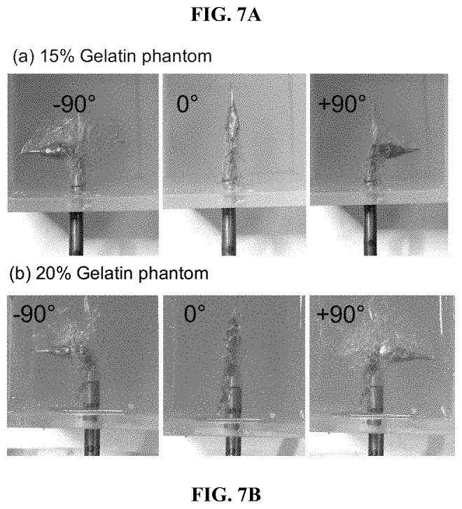

The ability of the robot to perform percutaneous procedure as well as distal bending at its wrist was evaluated in phantom models. Two different gelatin (Knox, USA) phantoms of 15% and 20% by weight were prepared to simulate biological tissues with different stiffness [36]. During each experiment, the SMA spring was heated to and maintained at 48° C. while the actuation cables were pretensioned and maintained at 1N. Then, the robot was inserted into the phantom by a linear actuator at a speed of 0.125 mm/s; 30 mm of the robot was inserted to the phantom in both experiments, as shown in the center pictures of FIG. 7. The results showed that the robot was capable of being inserted into the gelatin phantoms of different stiffness along a straight path without any noticeable sign of buckling. After the insertion process, heating of the SMA spring was stopped and it was allowed to cool down. The robot tip was then bent by 90° for a few cycles by actuating the cables connected to linear actuators on the proximal end. The results in FIG. 7 showed that the actuators were powerful enough to bend the wrist by the required bending angle even when the wrist was still in the phantom models. This confirmed that the robot was capable of producing distal bending even when a cavity, such as a pericardial cavity was only large enough to accommodate the needle but not the entire robot wrist, which remained in the stiffer biological tissues. Further, the silicone sheath stayed intact after the robot was removed from the insertion and distal bending tests in the phantom models.

### Example 8—Percutaneous Procedure Simulation Under Ultrasound Imaging

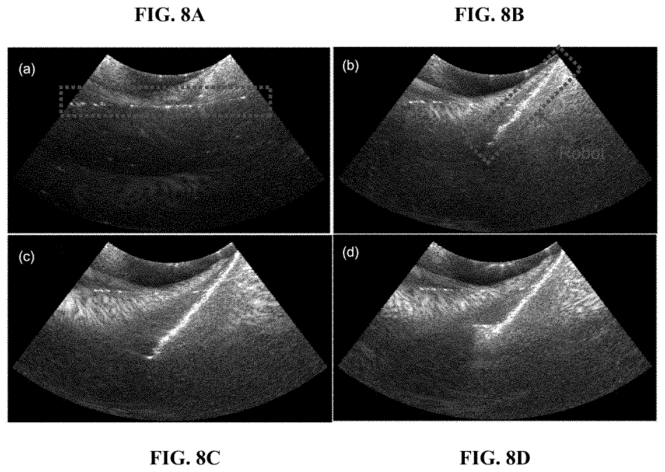

An experiment was performed in which the robot was inserted into a two-layer gelatin phantoms under ultrasound guidance. The top and bottom layers of the gelatin phantoms were 15% and 20% by weight, simulating the lipid and muscle layers in a human body, as shown in FIG. 8A. An ultrasound machine (Vantage 32 LE, Verasonics, Inc., USA.) with a probe (C5-2, Mindray, Inc., China) was used to provide the real-time imaging. The SMA spring was maintained at 48° C. and the actuation cables were pretensioned and maintained at IN during the robot insertion process. As shown in FIGS. 8A-8D, the robot was able to puncture through both layers of the gelatin phantom before finally making a distal bending motion in the imaging plane. The robot axis and tip were clearly visible during both the insertion and distal bending processes under ultrasound imaging. These results therefore confirmed the feasibility of the robot to be used in an ultrasound-guided pericardiocentesis.

### Example 9—Mechanical Design of a Robotic Cannula for Intracerebral Hemorrhage Evacuation (ICH)

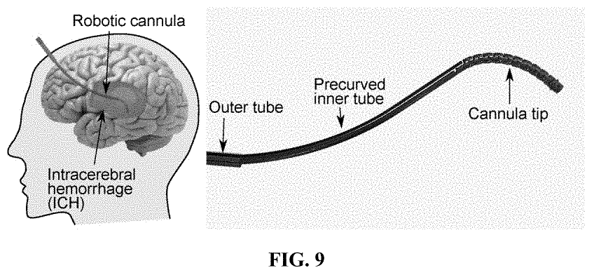

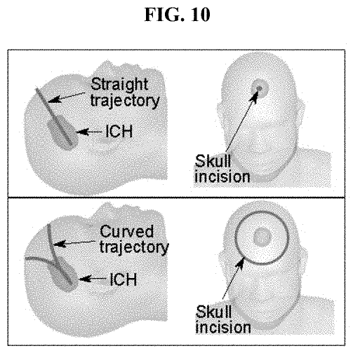

A neurosurgical device with pre-curved body and flexible tip was developed as a robotic cannula as shown in FIG. 9 to evacuate ICH under intraoperative image guidance (e.g. computed tomography (CT) or magnetic resonance imaging (MM)). A median ICH is often of an irregular shape, and has a volume of 16-75 ml [23]. Surgeons typically insert the cannula along its long axis to maximize ICH evacuation with minimal cannula pivoting. Contrary to many existing procedures that feature a straight path towards the ICH, the device of the invention targets through a curved trajectory that affords more options in terms of aesthetically favorable skull incision and safe insertion path to a surgeon while keeping the insertion direction along the long axis of the ICH, as shown in FIG. 10. The device of the invention comprises: A robotic cannula that offers a curved access towards the ICH and aligns with its long axis after the insertion. The curved trajectory and its associated safe insertion region can be identified by the surgeon based on preoperative CT or MR images to avoid critical brain structures. Using the device of the invention, the trajectory is limited to a single bend to minimize the insertion complexity and the risk of serious disruption to the healthy brain tissue during the insertion process.

The robotic cannula of the device of the invention is actuated at its distal end with around 90° bending capability in any direction, which ensures that the cannula reaches the peripheral ICH and covers at least 80% of the median volume of ICH to obtain clinically meaningful decompression. Advantageously, the dexterous manipulation of the distal tip is minimally coupled with the rest of its body to ensure minimal disruption to healthy brain tissue around the cannula body during the ICH aspiration process.

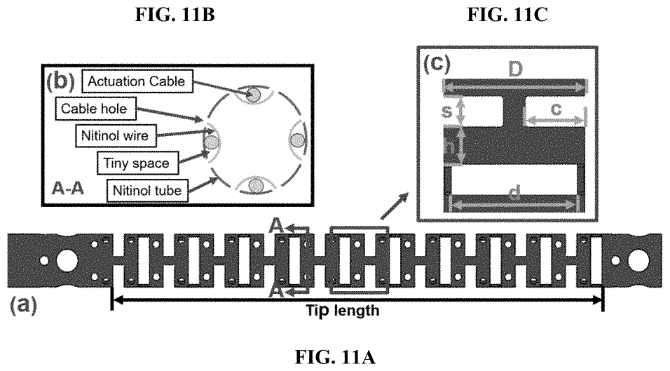

As shown in FIG. 11A, FIG. 11B, and FIG. 11C, the inner diameter, d, of the cannula of the device of the invention is at least 1.5 mm to house a suction tube of sufficiently large diameter that facilitates ICH aspiration. For comparison, the outer diameter of existing minimally invasive (single burr hole) ICH evacuation devices is in the range of 10 mm-15 mm due to the need to incorporate a neuroendoscope [24]. The outer diameter. D, of the robotic cannula of the device of the invention is about 3.0 mm. The cannula, when used under CT image guidance, therefore has the potential to reduce the trauma to healthy brain tissue due to the significant smaller size of the device inserted into the brain compared to conventionally used devices. The cannula of the device is biocompatible and sterilizable to eliminate the risk of infection.

The robotic cannula of the device of the invention consists of a pre-curved cannula body and a dexterous independently controlled flexible cannula tip and has a total of 3 active degrees of freedom (DoFs), including the translation of the cannula body and the 2-DoF bending of the cannula tip.

### Example 10—Pre-Curved Cannula Body

The concept of nested pre-curved tube was employed to achieve the curved insertion approach towards the ICH. The cannula body consisted of a straight outer tube with D=3.0 mm and d=2.6 mm, and a pre-curved inner tube of D=2.2 mm and d=2.0 mm, both of which were made of the biocompatible super-elastic Nitinol. The length of the outer tube was 100 mm while that of the inner tube was 220 mm. The outer tube provided a short straight channel extending from the robot base platform through the skull incision to the brain surface. The inner tube, with a pre-curved shape, covered a curved region based on its pre-curved shape once it was extended out of the constraining outer tube. The radius of curvature of the pre-curved inner tube was around 55 mm with a 45° bending angle. The curvature parameters were customized depending on the individual ICH case but were limited by the approximately 8% sustainable recoverable strain of Nitinol tube, as reported by the manufacturer (Peier Tech, China).

### Example 11—Flexible Cannula Tip

Because in existing procedures, neurosurgeons reach the peripheral ICH by pivoting the straight device about the site of skull incision, which creates significant disruption to the healthy brain tissues, and may have to use multiple insertion sites, the device of the invention was designed with an independently actuated flexible tip of the robotic cannula to allow exploration at the edges of an ICH without pivoting or multiple insertion to ensure safe and thorough ICH evacuation.

To this end, a flexible tip was made by creating notches in a Nitinol tube to introduce flexibility in the originally rigid straight super-elastic Nitinol tube. The cannula tip made of notched Nitinol tube exploited its seamless integration with the cannula body also made of Nitinol tube. While neurosurgeons typically do not favor a combination of rotation and bending DoFs since it could lead to disastrous outcome should any mechanical or electronic failure causes unexpected axial rotation in the system, the flexible cannula tip of the invention having a tip length of around 22 mm is capable of bending in both pitch and yaw DoFs to satisfy the workspace to completely cover a median volume ICH. An omnidirectional bending notched tube design was developed using symmetric or asymmetric notches. In one device, a symmetric rectangular notch pattern was used due to the large curvature required in the design (90° bending angle with only 22 mm bending length in any direction of the 3-dimensional space). A total of N=10 pairs of notches were equally and alternately distributed in two orthogonal planes. The critical geometric parameters of each notch (see FIG. 11C) included the cut depth c=0.93 mm, spacing height h=0.6 mm, and notch height s=0.5 mm. These parameter were chosen to satisfy the large bending curvature requirement. The notched tube design also allowed the flexible tip to have sufficient stiffness to be steered inside the ICH while being decoupled from its cannula body.

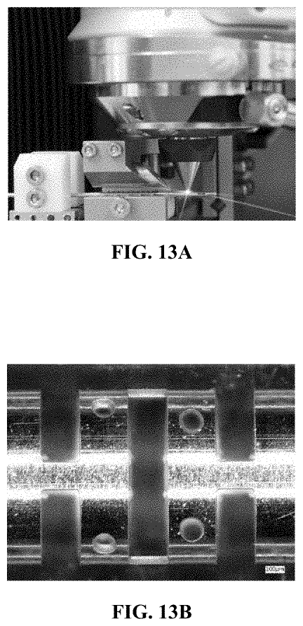

A cable-driven mechanism was used to actuate the flexible tip with two pairs of Stainless steel wire ropes (referred to as cables) with 0.21 mm diameter (Osaka Coat Rope Co., Ltd., Japan) responsible for pitch and yaw bending, respectively. They were routed along the periphery of the tube inside its lumen and terminated at the distal end of the tube. Several methods have been attempted to constrain the cables for cable-driven manipulator in general, such as combining concentric tubes with half-grooved channels to form complete cable channels and using 3D-printed fixtures embedded into selected notches. Because these approaches exhibit one or more undesirable features, such as increased outer diameter, reduced internal lumen diameter, and non-uniform curvature distribution along the flexible segment, in the device of the invention each actuation cable was constraint within the tiny space formed between a thin super-elastic Nitinol wire crossing and the tube inner wall. Each wire of 0.15 mm diameter was inserted between a pair of opposite holes on the tube wall (clearly seen in FIG. 11B and FIG. 13B) near the routing path of the cable. For each actuation cable a pair of these holes was made every two notches along the flexible tip to ensure the cable was robustly routed on the periphery of the tube throughout the bending process.

### Example 12—Actuation Unit

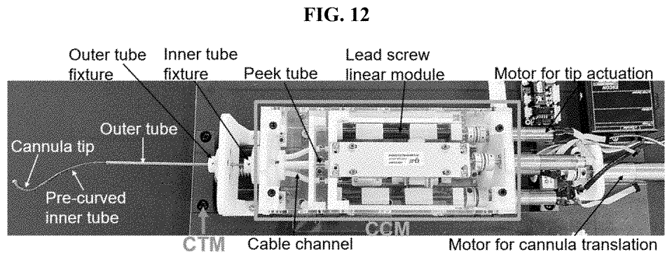

The actuation unit of the robotic cannula of the invention was mainly composed of a cable control module (CCM) and a cannula translation module (CTM) (FIG. 12). In the CCM, there were four DC Motors (DCX14, Maxon Precision Motors Inc., Switzerland) used to control the motion of four aluminum lead screw linear modules (Igus Inc., Germany) of OD 6 mm and pitch 1 mm. Four cables were respectively mounted on four load cells (Transducer Techniques, USA). Each load cell had a load capacity of 25 lbs and was fixed on the movable block of each linear module. The inner tube of the cannula was directly connected to the CCM, which was responsible of the 2-DoF bending motion of the flexible tip. The CTM consisted of an off-the-shelf aluminum linear module with OD 12 mm and pitch 3 mm (Igus Inc., Germany) and a DC motor (DCX22, Maxon Precision Motors Inc., Switzerland). It was used to translate the CCM and thus insert and retract the inner tube. The robot started with the inner tube completely withdrawn inside the straight outer tube. It was oriented in such a way that the ICH was in the same plane as the bending plane of the pre-curved inner tube. The CTM was then activated to extend the inner tube out of the outer tube, and into the brain. Once it reached the targeted point in the ICH, the CCM was activated to sweep around the ICH while performing hematoma aspiration.

### Example 13—Fabrication of the Cannula

The inner tube of the device of the invention went through a specific fabrication process. The notches were machined on the tip first before carrying out the shape setting for the tube body in a desired curvature and angle. Performing these fabrication processes on a single Nitinol tube offered a three-fold advantage. It eliminated the need for any assembly between the body and the tip (e.g. using heat shrink, glue or laser welding), inhibited any increase in the cannula's overall dimension that could result from the assembly method, and most importantly significantly improved the robot integrity and robustness. Because manufacturing by micro CNC milling, wire-electrode cutting machining, and laser micro-machining often induces heat-induced defects on the super-elasticity of the material and thus negatively impacts the bending behavior of a tip, a femtosecond laser micromachine (Starcut Tube L600 2+2 System, Coherent, Germany) was used to ablate the desired rectangular patterns on the tube. The laser micromachining system has a femtosecond laser source with wavelength of 1035 nm, pulsewidth from 300 fs-10 ps, and repetition rate of 50 MHz. A high-precision 3-dimensional lathe stage was used to translate and rotate the Nitinol tube during the laser cutting in order to keep the laser focus and power constant on the tube surface as shown in FIG. 13A. A portion of the flexible tip with the notches under the scanning electron microscope (SEM) is shown in FIG. 13B. The shape setting for the tube body was carried out by annealing at 510 degree for 20 minutes at a furnace.

### Example 14—Kinematics Validation of Ich Evacuation Device

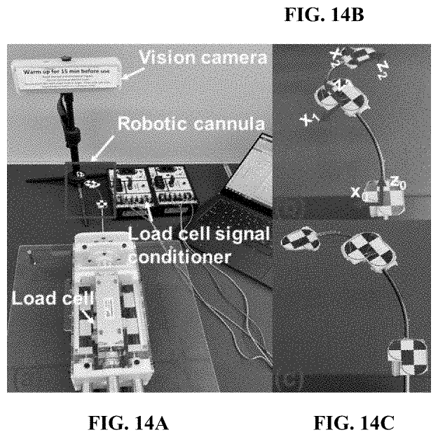

The experimental setup, shown in FIGS. 14A-14C, consisted of a robotic cannula system, load cells for every cable, and a vision camera (MicronTracker, Claron Technology, Canada). The camera was used to track the position and orientation information of the vision markers, which were attached to the distal and proximal ends of the cannula tip, and at the distal end of the outer tube, as shown in FIG. 14B. Cable tension was acquired by load cells fixed on the movable block in the CCM. Cable displacement was acquired by the motor encoders. A data acquisition system (Model 826, Sensoray Co., Inc., USA) was used to collect all the sensing data and the control program was implemented in Simulink (Mathworks Inc., USA).

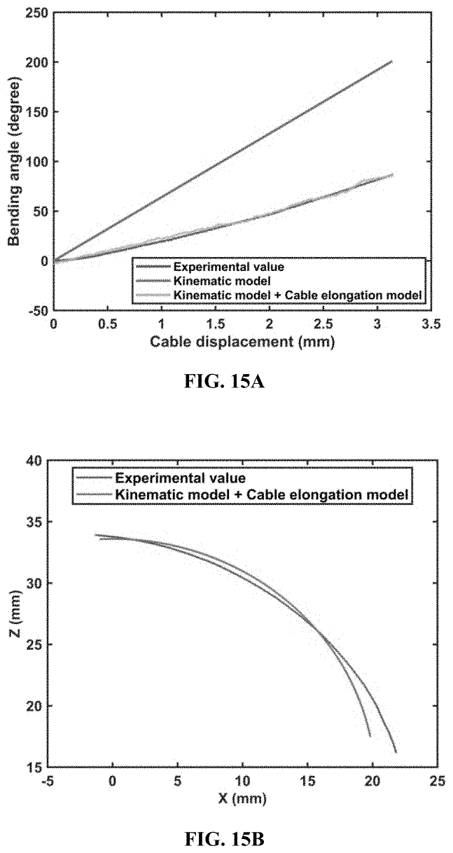

The kinematics of the cannula tip was significantly more complex than that of the cannula body, and thus was validated. After the inner tube was extended out to Ld=20 mm, the cannula tip's kinematics validation experiments were conducted by bending the tip from 0 to 90° in the counterclockwise direction, as shown in FIG. 14C. The cable was pulled from 0 to 2.822 mm at a constant velocity of 1 mm/min. The bending angle of the tip was plotted against the cable displacement in FIG. 15A using data from the experiment, and kinematic models with and without the CEM.

### Example 15—Decoupling Between the Cannula Body and Cannula Tip

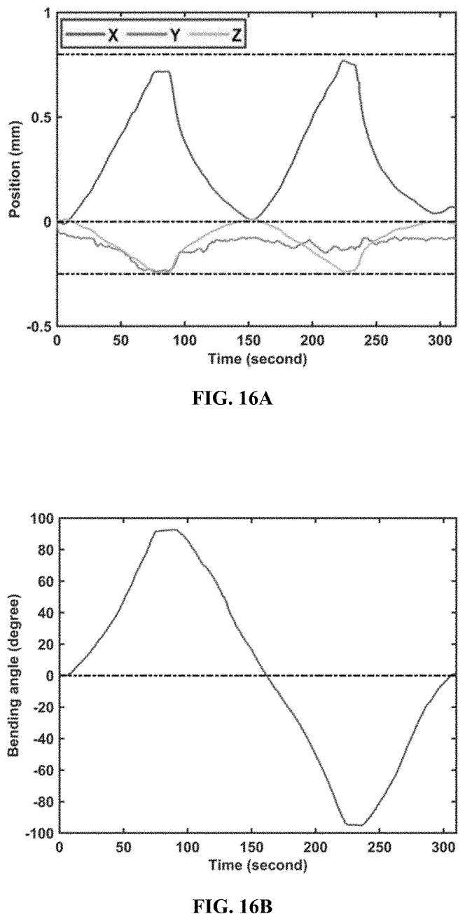

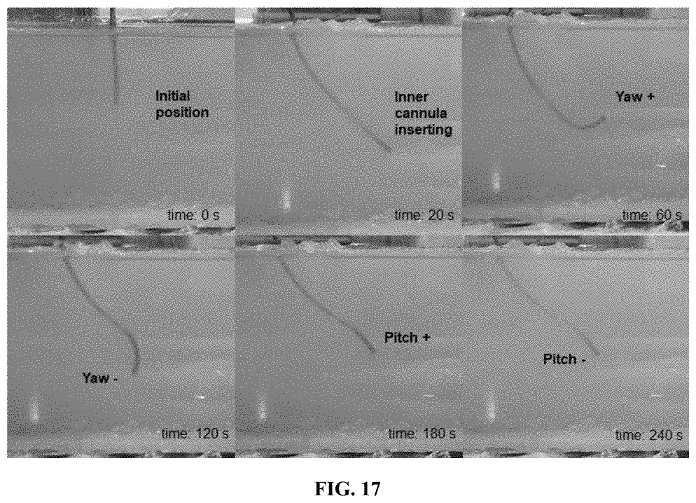

Since it was important that the cannula body did not produce significant motion during tip bending, the decoupling capability between the two parts of the cannula was determined. When the cannula body was at its extended state, the cannula tip was bent between 90° in the XZ plane. Then X, Y, and Z coordinates of the distal end of the cannula body and the bending angle of the cannula tip were plotted against time in FIGS. 16A and 16B, respectively. Considering the large cannula tip bending, submillimeter displacement of the cannula body was acceptable, confirming the decoupling capability between the cannula body and its tip.

### Example 16—Flexible Distal Segment

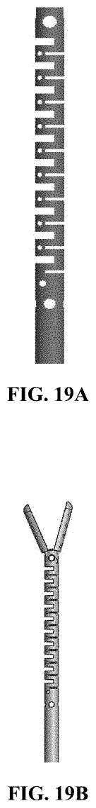

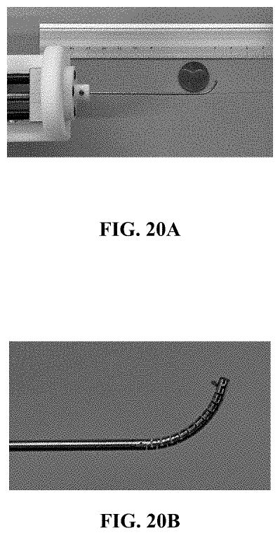

As shown in FIG. 20A and FIG. 20B, a neurosurgical instrument with 1.2 mm outer diameter, 1.0 mm inner diameter, and 10 mm length, consisting of a straight rigid body and a flexible distal segment was generated. The distal segment featured 20 lateral notches with the left notches having larger notch height than the right notches, allowing bending towards one (left) direction with large bending curvature up to around 150 m⁻¹ and bending angle of around 90° upon cable pulling. The notch patterns are shown in FIG. 19A and FIG. 19B. The small notch height on the right side minimized the structural stress during the large bending of the segment towards the left side and provided a mechanical limit to inhibit large bending angle towards the right side.

### Example 17—Fabrication of Flexible Neurosurgical Device

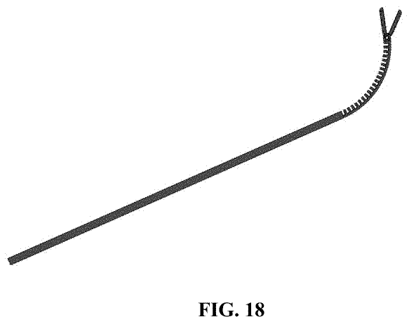

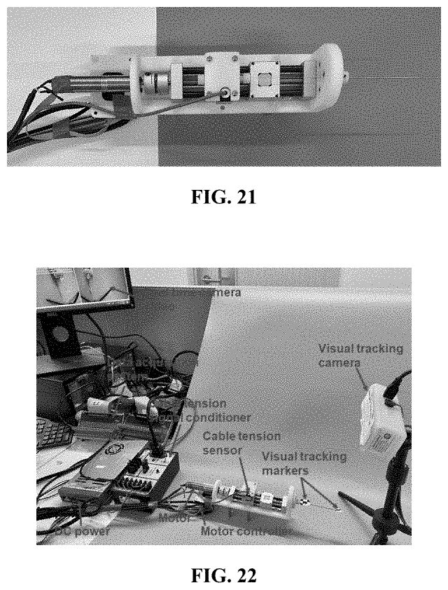

A femtosecond laser micromachining system was used to remove rectangular lateral notches with asymmetric patterns and dimensions at the distal section of a Nickel Titanium (Nitinol) tubing to introduce distal flexibility to the originally rigid tube. The geometrical parameters of each rectangular notch, including the lateral cutting depth, distance between consecutive notches, and notch height, were carefully selected to achieve the desired bending stiffness and bending curvature of the instrument. A stainless steel actuation cable with diameter 0.15 mm diameter made was terminated at the distal end of the flexible segment and routed along the lumen of the tubing before being connected to the control interface at the proximal end, as shown in FIG. 21. A pair of forceps, that was either actively or passively actuated, was integrated at the distal end of the instrument, as shown in FIG. 19B, to perform tumor biopsy at locations outside the line of sight due to the capability of the instrument to bend its distal segment, as shown in FIG. 18.

### Example 18—Experimental Setup for Device Testing

The experimental setup, shown in FIG. 22, consisted of the flexible neurosurgical instrument, a cable tension sensor (Transducer Techniques, USA), and a visual tracking camera (MicronTracker, Claron Technology, Canada). The flexible instrument was bent towards one direction via the cable-driven mechanism. The actuation cable, around 0.15 mm diameter, was terminated at the distal end of the flexible instrument, routed along the lumen of the instrument, and connected to the motorized actuation setup (consisting of a motor and a cable tension sensor). In some embodiments, the actuation setup was replaced by a custom-designed user control interface for manual operation. The camera was used to track the position and orientation information of the vision markers attached to the distal and proximal ends of the flexible instrument. The cable tension was measured by a cable tension sensor while the cable displacement was measured by the motor encoders integrated in the motor (DCX14, Maxon Precision Motors Inc., Switzerland). A data acquisition system (Model 826, Sensoray Co., Inc., USA) was used to collect all the sensing data and the motion control program was implemented in Simulink (Mathworks Inc., USA).

The stiffness and kinematic properties of the flexible instrument were evaluated using the experimental setup. The cable was pulled by the motor at a constant speed of 2 mm/min until the bending angle reached 70°, completing the loading process. Then the cable was released by the motor until the bending angle returned to 0°, thus completing the unloading process. During the entire loading and unloading processes, the cable tension and displacement were recorded, and plotted against their corresponding bending angle. The control system and sensing data acquisition system were run at a frequency of 10 Hz.

### Example 19—Stiffness Evaluation

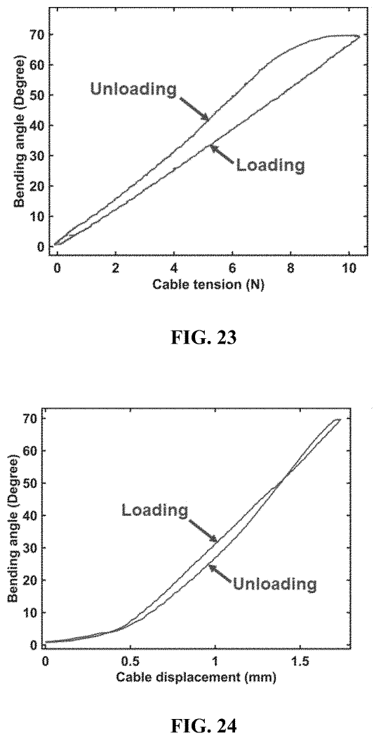

Cable tension and bending angle during both loading and unloading stages were measured and the relationship between cable tension and bending angle during both loading and unloading stages were plotted in FIG. 23. When the instrument was bent to 70°, the cable tension required was 10.37 N, proving a sufficiently high stiffness for motion in the brain and tissue manipulation.

### Example 20—Kinematics Evaluation

Cable displacement and bending angle during both loading and unloading stages were measured and the relationship between cable displacement and bending angle during both loading and unloading stages are plotted in FIG. 24. The cable was displaced for less than 2 mm for the entire range of motion (70°) of the flexible instrument. The kinematic performance demonstrated the dexterity of the instrument to satisfy the required bending angle of the instrument using a small amount of cable displacement.

We claim:

1. A stiffness tunable shape memory alloy (SMA)-based flexible wrist for surgical instruments that can be heat-activated and heat-controlled to achieve different stiffness and is cable/wire-driven to achieve a bending configuration.

2. A surgical instrument comprising the stiffness tunable SMA-based flexible wrist according to claim 1.

3. The surgical instrument according to claim 2 wherein the stiffness tunable SMA-based flexible wrist comprises a SMA spring and the surgical instrument further comprises a rigid arm, a needle, and an actuation interface, wherein the actuation interface is motorized or manual.

4. The surgical instrument according to claim 3, wherein the stiffness tunable flexible wrist further comprises a distal fixture and a proximal fixture both having two ends; wherein the distal fixture comprises a needle hub on one end and four prongs on the other end to interface with the needle and the SMA spring; and wherein the proximal fixture comprises four prongs on one end and four straight legs on the other end to interface with the SMA spring and the rigid arm.

5. The surgical instrument according to claim 4, wherein the distal fixture and the proximal fixture each further comprise at least one channel connected to the actuation interface, and wherein the rigid arm comprises a hollow tube comprising a heating tube and at least one rigid stainless steel tube containing at least one actuation cable connected to the actuation interface.

6. The surgical instrument according to claim 5, wherein the heating tube comprises a PTFE tube wound with a NiCr wire on its external surface.

7. The surgical instrument according to claim 3, wherein the SMA spring comprises a material that converts its material phase between a low stiffness martensite and a high stiffness austenite through temperature variation.

8. A method of using the surgical instrument according to claim 7 in a percutaneous or needle-based procedure, the method comprising:
heating the SMA spring using the heating tube;
inserting the surgical instrument into a body of a subject;
cooling the surgical instrument inside the body; and
actuating at least one actuator cable to bend the wrist of the surgical instrument to a desired angle.

9. The method according to claim 8, further comprising the step of pretensioning at least one actuator cable during the cooling step.

10. The method according to claim 9, wherein the percutaneous or needle-based procedure is pericardiocentesis.

11. A neurosurgical instrument comprising a straight or pre-curved instrument body and a flexible distal segment; wherein the neurosurgical instrument is monolithically fabricated from a single tubing.

12. The neurosurgical instrument according to claim 11, wherein the flexible distal segment comprises at least one symmetric or asymmetric notch parameters selected from notch shape, notch pattern, and notch dimension, wherein the flexible distal segment bends with a 1-degree of freedom (DoF) or 2-DoF motion.

13. The neurosurgical instrument according to claim 11, wherein the single tubing is fabricated from a medical grade materials selected from Nickel Titanium and Stainless Steel.

14. The neurosurgical instrument according to claim 12, further comprising additional flexible distal segments comprising at least one symmetric or asymmetric notch parameters selected from notch shape, notch pattern, and notch dimension, wherein the additional flexible distal segments bend with a 1-degree of freedom (DoF) or 2-DoF motion.

15. The neurosurgical instrument of claim 11 further comprising at least one end effector.

16. The neurosurgical instrument according to claim 15, wherein the at least one end effector is a biopsy forceps, scissors, a grasping forceps, a dissecting forceps, a suction tool, an irrigation tool, or an endoscopic camera.

17. The neurosurgical instrument of claim 11, further comprising a sheath made from metal or polymer, wherein the sheath is added to the flexible distal segment and increases the stiffness or compliance of the flexible distal segment.

18. The neurosurgical instrument of claim 11, further comprising a flexible sheath made from metal or polymer, wherein the sheath is integrated with the instrument to serve as actuation tendon or rod routing channel and hold sensors, wherein the sensors comprise a Fiber Bragg Grating (FBG).

19. The neurosurgical instrument of claim 11, further comprising sensors that detect external forces applied to the instrument at the tip or along the instrument body, measure the shape of the instrument, and measure position and orientation of the instrument.

20. A method of using the surgical instrument according to claim 17, comprising using the instrument in a neurosurgical lesion procedure.

21. The method according to claim 20, wherein the neurosurgical lesion procedure is selected from brain tumor removal, intracerebral hemorrhage removal, third ventriculostomy, and tumor biopsy.
